(19) United States
(12) Patent Application Publication
(10) Pub. No.: US 2023/0321683 A1
(43) Pub. Date: Oct. 12, 2023

(54) Method And Apparatus For Atomizing And Vaporizing Liquid

(71) Applicant: Abdulilah M. Mayet, Jeddah (SA)

(72) Inventor: Abdulilah M. Mayet, Jeddah (SA)

(21) Appl. No.: 18/092,331

(22) Filed: Jan. 1, 2023

Related U.S. Application Data
(60) Provisional application No. 63/362,744, filed on Apr. 9, 2022.

(51) Int. Cl. B05B 17/00

(52) U.S. Cl. CPC B05B 17/0646

(57) ABSTRACT

The present invention relates to a method and apparatus for atomizing and vaporizing liquid. An apparatus works as a fluid capsule and pumps air saturated with liquid and carrying a mist of tiny droplets sized at or less than about 2 μm in diameter. The pump may have a vibrator attached to the liquid reservoir to cause liquid to move up through the outlet valve and out through an orifice located on the top of the capsule. One or more droplets of liquid ejected from the ejector may be heated and vaporized into the air. The design of the fluid capsule ensures that there is no larger droplet size of liquid that is allowed to come out. The fluid can be air-freshener, air-sanitizer, disinfectant, insect repellent or any other liquid.

### CROSS-REFERENCE DATA

The present application claims a priority date benefit from my co-pending U.S. Provisional Patent Application No. 63/362,744 with the same title filed Apr. 9, 2022 and incorporated herein by reference in its entirety.

### FIELD OF INVENTION

The present invention generally relates to a to an apparatus and method for atomizing and vaporizing liquid.

### BACKGROUND ART

Commercial air fresheners generally fall into two categories: (i) mechanical atomizers, which use a mechanical motor operated by a power source to push a valve and allow compressed perfume inside a can to be released; and (ii) perfume evaporator and burners, which use an open flame to evaporate perfume carried by an additive such as wax or other combustible material.

US20170368273A1 describes techniques for optimal amount of airflow regulation under all conditions of aerosol delivery. Prior aerosol delivery devices include designs aimed at achieving greater control over laminar flow and/or flow velocity and volume of aerosolized air for improved aerosol delivery to user airways; greater and longer expansion of user airways, such as with positive pressure, so that airways are more receptive to receiving aerosolized medicament formulations; selective targeting of aerosols to different regions of the airways, such as the upper airways, lower airways, and or providing systemic delivery through the pulmonary route; accommodation of more medicament formulations that have potential for aerosolization; including liquids, suspensions and solids, droplets and particles, of varying sizes, shapes, weights, viscosities, and flow dynamic properties.

US20160216712A1 discloses methods for the generation and application of an ultrasonically generated aerosol for uses including but not limited to the sanitization, detoxification, disinfection, high-level disinfection, or sterilization of one or more areas and the surfaces in those areas, as well as the delivery of other types of liquid agents, for various purposes to one or more areas, and without limitation, the surfaces in those area(s).

Commercial air fresheners have drawbacks. For example, commercial air fresheners may waste energy and may cause pollution. Mechanical atomizers can consume a lot of power while operating the motor and spray the perfume in form of droplets that do not dissolve into the air due to the high surface tension and low vapor pressure of the fluid. Perfume evaporator and burners may cause air pollution due to the flame and carbon dioxide generation. Additionally, perfume evaporators and burners may change the chemistry of the perfume due to the high temperature, which may be harmful if inhaled.

Another drawback of commercial air fresheners is the short duration of the air freshening effect of the products. Commercial air fresheners generally deliver large droplets of fluid into the air, which can fall to the ground due to gravity, and settle to the ground, and result in slow perfume dispersal requiring even more perfume to be delivered. Additionally, the current commercial electrical air fresheners are often bulky and inefficient devices, which consume a large amount of energy and waste fragrance by dispensing large droplets of fragrance into the air. Thus, there is a need for an improved air freshening device.

Further, the prior art does not disclose a feature of providing a fluid capsule having the bubble caps to prevent larger droplets sizes from ejection and assure only the fine mist with micrometer diameter and existence of valves to prevent fluid spilling.

### SUMMARY OF THE INVENTION

The following summary is an explanation of some of the general inventive steps for the device, method and construction in the description. This summary is not an extensive overview of the invention and does not intend to limit the scope beyond what is described and claimed as a summary.

The present invention in some embodiments thereof, generally relates to a method and apparatus for atomizing and vaporizing liquid. An apparatus works as a fluid capsule and pumps air saturated with liquid and carrying a mist of tiny droplets sized about 2 μm or less in diameter. The pump may have a vibrator that vibrates the liquid reservoir and causes liquid to move up through the outlet valve and out through an orifice located on the top of the capsule. The one or more droplets of liquid ejected from the ejector may be heated and vaporized into the air. The design of the fluid capsule ensures there is no larger droplet size of liquid come out. The fluid can be air-freshener, air-sanitizer, disinfectant, insect repellent or any other liquid.

According to on embodiment, the disclosed device addresses the limitations of the prior art by disclosing a feature of providing a fluid capsule having bubble caps to prevent larger droplets sizes from ejection and ensure that only fine mist with droplets having a diameter at or less than 2 μm is allowed to flow with the air emanated from the device. Provides also are valves to prevent fluid spilling or leakage when the device is off.

### BRIEF DESCRIPTION OF THE FIGURES

FIG. 1 of the diagrams illustrates an exemplary fluid atomizer according to one embodiment.

FIG. 2 of the diagrams illustrates an exemplary fluid atomizer system according to one embodiment.

### DETAILED DESCRIPTION OF THE INVENTION

Hereinafter, the preferred embodiment of the present invention will be described in detail with reference to the accompanying drawings. The terminologies or words used in the description and the claims of the present invention should not be interpreted as being limited merely to their common and dictionary meanings. On the contrary, they should be interpreted based on the meanings and concepts of the invention in keeping with the scope of the invention based on the principle that the inventor(s) can appropriately define the terms in order to describe the invention in the best way.

It is to be understood that the form of the invention shown and described herein is to be taken as a preferred embodiment of the present invention, so it does not express the technical spirit and scope of this invention. Accordingly, it should be understood that various changes and modifications may be made to the invention without departing from the spirit and scope thereof.

Fragrance delivery systems and methods are generally described herein. The appliance is provided for volatilizing and atomizing fluid. The device includes a high-frequency piezoelectric transducer to volatile the fluid. Fine-size droplets which are floating in the air will pass the bubble caps, while large droplets size will fall back into the fluid reservoir. The heated forced airflow will eject the fragrance through the exhaust orifice with a higher concentration. The apparatus may include valves to prevent fluid from spilling from the device. A heater may be mounted next to the air pump configured to eject evaporated fluid and small droplets of liquid from the orifice of the ejector.

The apparatus may include a vibrator coupled to the ejector, which is configured to provide longitudinal vibration to the fluid. The apparatus may include a power source to provide power to the vibrator. In at least one embodiment, the apparatus is configured to be inserted into a decorative object.

An ejecting apparatus may be inserted into a reservoir containing liquid. The ejecting apparatus may be longitudinally vibrated to cause fluid to be displaced longitudinally through the bottom orifice of the ejector and expelled from the top orifice of the ejector. The fluid ejected from the ejecting apparatus may be heated to vaporize the ejected fluid into the air.

According to one embodiment, an apparatus including an ejector configured to eject one or more droplets of liquid from an orifice at the top of the ejector is inserted into a liquid reservoir is disclosed. Vibration may be applied to the ejector, which causes liquid to pump into the ejector and out through the orifice at the top of the ejector. A heater positioned above the orifice of the ejector may heat the ejected droplets to atomize and vaporize the liquid faster than at ambient temperature. The heating can occur by the droplets impacting the hot surface or experiencing a higher temperature in the air as the droplet goes through the hot air.

It is an objective of the invention is to dissolve released fragrance in the air, which will extend the air freshening effect. Another objective is to manufacture a device that is more sustainable and more cost-effective than current commercial air fresheners. An additional purpose of the instrument is to release the scent into the air without changing its chemical composition.

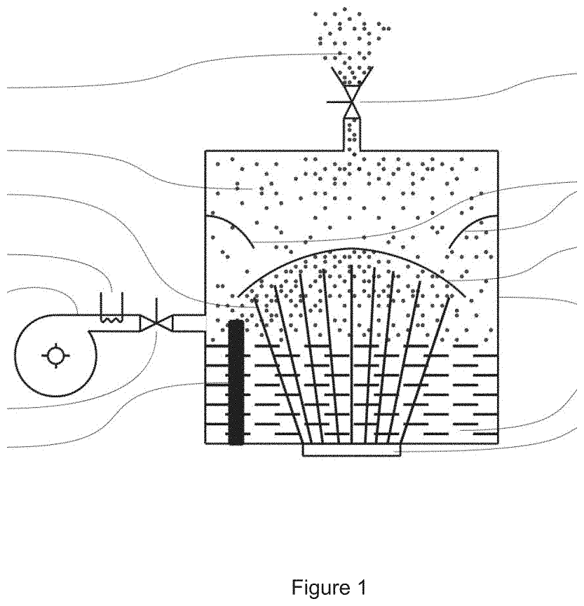

FIG. 1 of the diagrams illustrates an exemplary fluid atomizer according to one embodiment. The fluid atomizer, as shown, comprises an orifice at a top portion isolated with a valve, the capsule is configured to pump air saturated with fine mist droplets of liquid from the orifice, a heater to raise the air temperature to increase the fluid solubility in the injected air, bubble caps provided thereto to prevent larger droplets sizes from ejection and ensure only the fine mist with diameter less than 2 μm to flow with the air, a vibrator mounted undemeath the fluid reservoir to assure maximum fluid flow rate and an airtight sealant structure to prevent fluid leakage or flow at off condition.

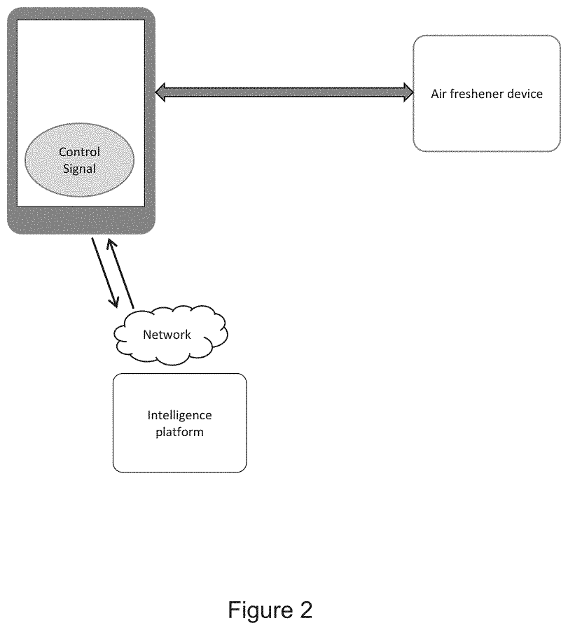

Referring to FIG. 2 of the diagrams, illustrated as an exemplary fluid atomizer system according to one embodiment comprising a mobile-operated atomizer, such as the one illustrated in FIG. 1.

Generally, the construction of the fluid capsule for atomizing and vaporizing liquids may be such that it is provided an orifice at a top portion isolated with a valve, the capsule is configured to pump air saturated with fine mist droplets of liquid from the orifice. Further, a heater may be provided to raise the air temperature to increase the fluid solubility in the injected air. A bubble cap is provided thereto to prevent larger droplets sizes from ejection and ensure only the fine mist with diameter of less than 2 μm to flow with the air. A vibrator is mounted underneath the fluid reservoir to assure maximum fluid flow rate, and an airtight sealant structure is used to prevent fluid leakage when the device is not operating.

It is anticipated that a microcontroller to control the operation of the device to ensure the demanded flow rate is provided.

It is anticipated that a diaphragm air pump to blow air into the apparatus is provided to the capsule.

It is anticipated that a piezoelectric plate may be provided as the vibrator.

It is anticipated that a halogen lamp heater is provided as a heater to increase the fluid temperature to improve atomization.

It is anticipated that an isolation mechanism to separate large droplet sizes of liquid to remove them from the air flow is provided.

According to one embodiment, the microcontroller has a power source in the form of a solar cell, and a timing device configured to control the time and duration of liquid ejection from the ejector.

According to one embodiment, a plurality of valves in the airflow path may have an orifice at a top portion, wherein an inlet valve isolates the capsule from the air pump and the heater, an outlet valve isolates the capsule from the environment and has a top orifice, and a heater mounted in the air path between the air pump and the inlet valve to increase the liquid temperature to improve misting, wherein the fluid capsule only pumps air saturated with fluid mist either dissolved or at tiny droplets size of 2 μm.

It is anticipated that the outlet valve locks the outcome hole while the device is off to prevent liquid leakage.

It is anticipated that a mobile device, preferably a smartphone, is provided and equipped with a suitably trained artificial intelligence algorithm to control the operation of the capsule to spray the fluid mixture according to the client's demand and their mood.

It is further anticipated that the device comprises at least ten scent ingredient chambers.

Although a preferred embodiment of the present invention has been described for illustrative purposes, those skilled in the art will appreciate that various modifications, additions and substitutions are possible, without departing from the scope and spirit of the invention as disclosed in the accompanying claims. Such alterations are herewith anticipated.

### INDUSTRIAL APPLICATION

The invention is applicable in the manufacture of air freshener devices.

What is claimed is:

1. A fluid capsule for atomizing and vaporizing liquids, the capsule comprising:
an orifice at a top portion isolated with a valve, the capsule is configured to pump air saturated with fine mist droplets of liquid from the orifice;
a heater to raise the air temperature to increase the fluid solubility in the injected air;
bubble caps configured to prevent droplets above 2 μm in size from ejection and allow the fine mist with droplets having a size of 2 μm or less to flow with the air;
a vibrator mounted underneath the fluid reservoir to maximize fluid flow rate; and
an airtight sealant structure to prevent fluid leakage while the fluid capsule is not operating.

2. The fluid capsule of claim 1, further comprising a microcontroller to control the operation of the device to provide a required flow rate.

3. The fluid capsule of claim 1, further comprising an air pump configured to blow air into the apparatus.

4. The fluid capsule of claim 3, wherein the air pump is a diaphragm air pump.

5. The fluid capsule of claim 1, wherein the vibrator is a piezoelectric plate.

6. The fluid capsule of claim 1, wherein the heater is a halogen lamp heater.

7. The fluid capsule of claim 1, further comprising a fluidic capsule adapted to operate with the airflow system.

8. The fluid capsule of claim 1, further comprising an isolation mechanism of the large droplet sizes of liquid to separate them from the airflow.

9. The fluid capsule of claim 1, wherein the vibration is applied at a frequency at or above 1 megahertz.

10. The fluid capsule of claim 1, wherein the microcontroller has a power source sufficient to provide power to the vibrator.

11. The fluid capsule of claim 1, further comprising a timing device in the micro-controller, the timing device is configured to control the time and duration of liquid ejection from the ejector.

12. The fluid capsule of claim 1, further comprising a solar cell configured to charge the power source in the microcontroller.

13. The fluid capsule of claim 1, further comprising:
a plurality of valves in the airflow path having an orifice at a top portion, wherein the plurality of valves further comprise:
an inlet valve to isolate the capsule from the air pump and the heater;
an outlet valve to isolate the capsule from the environment and has a top orifice;
wherein the heater is mounted in the air path between the air pump and the inlet valve,
wherein the fluid capsule pumps air saturated with fluid mist comprising droplets sized at or less than 2 μm.

14. The fluid capsule of claim 13, wherein the outlet valve locks the outcome hole to prevent liquid leakage while the fluid capsule is not operating.

15. The fluid capsule of claim 1, wherein a mobile device is provided equipped with a suitably trained artificial intelligence algorithm to control the operation of the capsule to spray the fluid mixture according to the client's demand.

16. The fluid capsule of claim 1, wherein the device comprises multiple fragrances, sanitizers, and active ingredient chambers.

17. The fluid capsule of claim 1 further comprising a wireless charger.
